(19) United States
(12) Patent Application Publication
(10) Pub. No.: US 2024/0108344 A1
(43) Pub. Date: Apr. 4, 2024

(54) Non-Invasive Wound Closure Device

(71) Applicant: Jamil Mogul, Santa Clara, CA (US)

(72) Inventor: Jamil Mogul, Santa Clara, CA (US)

(21) Appl. No.: 18/088,401

(22) Filed: Dec. 23, 2022

Related U.S. Application Data
(60) Provisional application No. 63/413,027, filed on Oct. 4, 2022.

(51) Int. Cl. A61B 17/08

(52) U.S. Cl. CPC A61B 17/085; A61B 2017/088

(57) ABSTRACT

A non-invasive wound closure device without use of any needles, sutures, staples or tissue-adhesives, to reapproximate the everted edges of a wound of a patient, has two side strips each containing a customized bandage adhered to either side of the wound that is closed by mating and mechanistically interlocking the two side strips, that can also be reopened by mechanistically unlocking and separating the mated side strips, and that can be then reclosed by repeating the closure process. Each side strip has interlocking feature(s) to securely lock them in place with or without an additional interlocking strip or an interlocking slider and has an integral adhesive-bandage layer on the bottom to adhere to the patient's skin. The device may be made in various lengths to encompass a variety of wounds or in a plurality of segmented side strips so as to align the device along a curved wound.

### CROSS REFERENCE TO RELATED APPLICATIONS

Applicant claims priority of provisional application 63/413,027, filed Oct. 4, 2022, for “Wound Closing Device”.

### BACKGROUND

### Field of the Invention

The present invention relates to an apparatus that reapproximates the everted edges of a wound or surgical cut without a need for sutures or staples, or any invasive surgical tool or needle and often without any anesthetics, and that allows the practitioner to inspect, reinspect, reoperate on, if necessary, and reclose the wound or cut without any invasive surgical tool or needle and often without any anesthetics.

### Prior Art

A prevalent practice to reapproximate the edges of a wound or surgical cut is by cleansing, anesthetizing, irrigating, and debriding the wound or cut as necessary and by using an absorbable or nonabsorbable suture and surgical tools including a needle. Suturing techniques are as follows: continuous, Interrupted, deep, buried, purse-string, and subcutaneous suturing. Typically, while a patient is under a general or local anesthesia, the surgical tool's needle with a suture is inserted into one side of the wound or cut and out through the wound or cut itself, and the suture is extended through the full depth of the dermis. Then the needle is re-inserted at the same level in the opposite side of the wound or cut and emerges out of the skin the same distance from the edge of the wound or cut as the insertion. Often during the suturing process, a surgical tool, such as forceps, or a gauze pad, or finger is used in order to evert the edges of the wound or cut so that the germinating layers of the skin are in contact with one another.

Another practice to reapproximate the edges of a wound or surgical cut is by using a surgical stapler. In this practice, the wound or cut is cleansed, anesthetized, irrigated, and debrided as necessary. From one end of the wound or cut, the edges of the wound or cut are everted by using forceps, gauze pads, or by pinching the nearby skin between a thumb and forefinger. A surgical stapler is gently placed across the wound or cut by making sure that the center of the head of the stapler is perpendicular to and over the center of the wound or cut. Then by squeezing the handle of the stapler, a staple is automatically placed into the wound and bent to a proper configuration. More staples are placed by moving the stapler along the wound's or cut's length and by further squeezing the stapler handle in order to continue to reapproximate the edges of the wound or cut while making sure that each staple is placed to a certain distance from its predecessor, as needed to achieve proper apposition of the edges along the entire length of the wound or cut.

Yet another practice to reapproximate the edges of a wound or surgical cut is by using a tissue adhesive. In this practice, the wound or cut is cleansed, anesthetized, irrigated, and debrided as necessary. The edges of the wound or cut are everted and held together with forceps, gauze pads, or fingers, and an adhesive-carrying plastic tube is squeezed to expel droplets of the adhesive through a cotton-tipped applicator at the end of the tube. The adhesive is applied in at least three to four thin layers along the length of the wound's or cut's surface and the applied adhesive is extended to a certain distance, as appropriate, from each side of the wound or cut. The edges of the wound or cut are supported and held together for a certain amount of time while the adhesive dries. Once the adhesive has dried completely, the closure of the wound or cut is further protected with a nonocclusive bandage.

Still another practice to reapproximate the edges of a wound or surgical cut is by using a medical adhesive tape or strip with a peel-away backing. In this practice, the wound or cut is cleansed, anesthetized, irrigated, and debrided as necessary. After the wound has been dried, a liquid adhesive is applied to the skin adjacent to the wound or cut to increase adhesion of the tape, which is handled with gloved hands. With the backing still attached, the tape is cut to the desired length or long enough to allow a certain distance, as appropriate, of overlap on each side of the wound or cut. The tape is gently removed from its backing with forceps by pulling straight back while making sure that the tape is not curled. Half of the tape is placed securely at the midportion of the wound or cut and the opposite edge of the wound or cut is gently but firmly apposed to the counterpart edge while applying the second half of the tape next and holding the wound edges as close together as possible and at equal height. More tapes are applied by bisecting the remainder of the wound or cut so that the wound or cut is completely apposed without totally covering the entire length of the wound or cut. Also, cross tapes are placed to add support to the tape ends.

Still another practice to reapproximate the edges of a wound or surgical cut is by using a plastic adhesive strip with pull tabs, a peel-away backing, and a middle structure that forms a living hinge and contains one-way ratchet locks. In this practice, the wound or cut is cleansed, anesthetized, irrigated, and debrided as necessary, the pull tabs are pinched toward the center and the backing is lifted off, and while holding it by the tabs, the adhesive strip is centered across the wound, placed on the skin, and the strip is smoothed on either side. Multiple strips, in parallel, are placed a certain distance apart until the length of wound or cut is covered. Then once the length of the wound is covered, the tabs on each strip are pulled in opposite directions. Doing so allows the hinge and ratchet locks to function so as to move and lock in place the strip's sides. Then, the tabs are cut off with surgical scissors and the closed wound or cut is covered.

Still another practice to reapproximate the edges of a wound or surgical cut is by using an adherable strip of mesh and another adherable strip of mesh that has multiple hooks on its one face. In this practice, the wound or cut is cleansed, anesthetized, irrigated, and debrided as necessary. The mesh strip is placed longitudinally on and adhered to one side of the wound or cut while the other mesh strip with the hooks is placed longitudinally on and adhered to the other side of the wound or cut while hooks facing the mesh strip. Then, the hooks are engaged into the mesh after the opposite edge of the wound or cut is apposed to the counterpart edge while holding the wound edges as close together as possible. After this, the closed wound or cut is covered.

Still another practice to reapproximate the edges of a wound or surgical cut is by using two adherable strips, each longitudinally containing a flexible magnet. In this practice, the wound or cut is cleansed, anesthetized, irrigated, and debrided as necessary. One magnetic strip is placed longitudinally on and adhered to one side of the wound or cut while the other magnetic strip is placed longitudinally on and adhered to the other side of the wound or cut. Then, the magnetic strips are joined together to magnetically mate them after the opposite edge of the wound or cut is apposed to the counterpart edge while holding the wound edges as close together as possible. After this, the closed wound or cut is covered.

### The Problems Addressed

Sutures used for closing a wound or surgical cut are made of synthetic or natural materials. Certain suture materials may cause an allergic reaction to a patient. Some sutures are absorbable while others need to be removed once sufficient wound healing has occurred. Wound infection occurs in skin surgeries, depending upon the type of procedure, type and location of the wound or cut, and patient factors. Complications after the procedure include increasing redness, swelling, fever, pain around the wound or cut, or pus or discharge from the wound or cut. Additionally, sutures pose a risk of needle-stick injury to the patient and also to the practitioner.

Another complication of suturing is wound or cut dehiscence, which occurs when a wound or cut heals properly and stiches are removed, the sutures around its edges stay intact while a granulation tissue or granuloma starts forming. It is, among other factors, often due to utilization of an inappropriate suture knot, tying the suture knots too loosely, employment of an improper suture material, over-tightening or misplacement of the suture, and/or an incorrect suture technique. A clean reopened wound or cut can be re-stitched but an infected wound or cut is often left to heal. Absorbable sutures can sometime extrude through the skin as they dissolve. Late complications include scars or hypertrophic scarring and keloid formations, which may be due to either improper suturing with excess tension or lack of eversion of the edges. Other complications include stitch marks and wound necrosis.

Stapling, which is much faster than suturing also causes scars in the skin similar to sutures, but the scars resulting from staples are often more pronounced. Patient acceptance and comfort and wound infection and dehiscence are similar with staples-closed wounds or cuts and sutured wounds or cuts. However, removal of staples can be somewhat more uncomfortable than removal of sutures. Also, cosmesis suffers, especially, if the staples are left in too long.

Wounds or surgical cuts closed with tissue adhesives have been found to have rates of healing and complications that are equivalent to those of staples-closed wounds or cuts and sutured wounds or cuts. Additionally, if an excessive amount of adhesive is applied too quickly, the patient can experience and sustain a local burn from the heat of polymerization, and if the adhesive is washed or soaked, it will peel off in a few days before the wound is healed. Also, if the adhesive contacts the clinician's gloved fingers, the glove may adhere to the patient's skin. There is a slightly higher risk for wound or cut dehiscence in wound closures with the adhesives than with sutures. Tissue adhesives cannot be used for closing wounds or cuts with a high-tension skin, nor can they be used near the eyes, over or near joints, on moist or mucosal areas, or on wounds or cuts under a highly static or dynamic skin tension.

Adhesive tapes or strips used for closing wounds or surgical cuts often lead to inferior cosmetic results, and premature tape separation may often occur. Other complications include skin blistering if no cross-strips are used or the tape is stretched too tightly across the wound, and hematoma may occur if hemostasis is inadequate. Also, tape may loosen prematurely over shaved areas as hair grow back.

As they are used across a wound, the adhesive strips with a hinge, pull tabs and ratchet locks, which seem functionally and operationally similar to the adhesive tapes or strips that are commonly used for wound closures, may have similar complications. Also, the adhesive strips with a hinge, pull tabs and ratchet locks do not have provisions for reinspection of, or reoperation on and reclosure of the wound as the tabs are cut off after closing the wound. Even if the tabs are not cut off, the ratchet locks are one-way that does not allow the sides of the strip placed across the wound to be moved back or brought to the strip's initial state.

Similarly, the mesh and hooks strips cannot be disengaged easily for reinspection of or reoperation on the wound without distorting or breaking the hooks or receptacle mesh holes. Hence the strips cannot be effectively re-engaged to re-close the wound.

The magnetic strips that are not yet on the market and that seem functionally and/or operationally similar to the mesh and hooks strips for a wound closure may be unreliable as they may not stay mated due to the patient's movement or the high-tension skin's static or dynamic force that is greater than the magnet's attractive force.

### A Solution to the Problems

To address the issues and complications mentioned above, a non-invasive wound closure device is designed to reapproximate the everted edges of a wound or cut often without any anesthetics faster, more simply and safely, and less painfully than staples, sutures, tissue adhesives or adhesive tapes, or the abovementioned non-invasive strips with pull tabs/hinge/ratchet locks, mesh and hooks strips, or magnetic strips.

### BRIEF SUMMARY OF THE INVENTION

In accordance with the present invention, there are four embodiments of the invention, which are briefly stated below.

In the first embodiment, the wound closure device consists of two side strips, either of which, on its bottom, is attached to a custom bandage and contains on its top an offset protrusion along the entire length, which has alignment holes, and which forms a wedge tongue when the protrusions are joined together, and a slider strip, which, longitudinally, has a transversely blind slot to form a matching wedge groove.

The custom bandage is a dimensionally modified version of a typical adhesive bandage with a peel-away backing. The modification is made to lengthen the fabric absorbent to the length of the side strip and to offset the fabric absorbent to one side.

To reapproximate a wound that has been medicinally prepped, once its backing is peeled off, the side strip is placed on and adhered to one side of the wound while making sure that the protrusion is parallel and adjacent to the wound's edge. For the other side of the wound, the same is done using the other side strip while making sure that its protrusion faces the protrusion of the other placed side strip.

Now, both placed side strips are pressed down and brought toward each other to evert the edges of the wound and mated by aligning the protrusion holes and joining the protrusions together and then the slider strip is installed by inserting the joined protrusions into the slider strip's slot, and by sliding the slider strip all the way, which keeps the two side strips mated, resulting in wound closure.

To reinspect and/or reoperate on the wound, the slider strip is slid out, which will separate the mated side strips and thereby open an access to the wound. And to reclose the wound, both placed side strips are re-mated as mentioned above.

In the second embodiment, the wound closure device consists of two side strips, either of which has an offset protrusion on its top along its entire length and is attached at its bottom to the custom bandage. The protrusion of the first side strip has an angled slot on the top and a straight slot on the side and is called a receiving protrusion, while the protrusion of the second side strip has a flex-hinged flap latch on the top and a straight rib on the side and is called a latching protrusion.

To reapproximate a wound that has been medicinally prepped, once its backing is peeled off, the first side strip is placed on and adhered to one side of the wound while making sure that the receiving protrusion is parallel and adjacent to the wound's edge. For the other side of the wound, the same is done using the second side strip while making sure that the latching protrusion faces the receiving protrusion.

Now, both placed side strips are pressed down and brought toward each other to evert the edges of the wound and mated by fully inserting the straight rib into the corresponding straight slot; and then while holding the mated side strips together, the flap latch is placed by inserting the angled rib into the corresponding angled slot, which keeps the two side strips mated, resulting in wound closure.

To reinspect and/or reoperate on the wound, the latch is lifted to disengage it, which will separate the mated side strips and provide an open access to the wound. And to reclose the wound, both placed side strips are re-mated as mentioned above.

In the third embodiment, the wound closure device consists of two side strips, either of which has an offset protrusion on its top along its entire length and is attached at its bottom to the custom bandage. The protrusion of the first side strip, along its entire length, contains a linear opening and a cross-section whose shape looks like the letter G and is called a G-receptacle protrusion. The protrusion of the second side strip, along its entire length, contains, on its one side, an extended rib with a flanged end, and is called an engaging protrusion.

To reapproximate a wound that has been medicinally prepped, once its backing is peeled off, the first side strip is placed on and adhered to one side of the wound while making sure that the G-receptacle protrusion is parallel and adjacent to the wound's edge. For the other side of the wound, the same is done using the second side strip while making sure that the engaging protrusion faces the G-receptacle protrusion.

Now, both placed side strips are pressed down and brought toward each other to evert the edges of the wound and mated by fully inserting the engaging protrusion's extended rib into the corresponding linear opening of the G-protrusion, which keeps the two side strips mated, resulting in wound closure.

To reinspect and/or reoperate on the wound, the linear opening of the G-receptacle protrusion is widened with fingers or a pin to disengage the extended rib; and the mated side strips are manually separated, which will provide an open access to the wound. And to reclose the wound, both placed side strips are re-mated as mentioned above.

In the fourth embodiment, the wound closure device consists of two side strips, either of which is attached at its bottom to a custom bandage and on its top, the side strip, along its entire length, contains an offset integral standard zipper-teeth chain, and a standard zipper slider for interlocking zipper-teeth chain.

To reapproximate a wound that has been medicinally prepped, once its backing is peeled off, the side strip is placed on and adhered to one side of the wound while making sure that the zipper-teeth chain is parallel and adjacent to the wound's edge and. For the other side of the wound, the same is done using the second side strip while making sure that its zipper-teeth chain faces the zipper-teeth chain of the other side strip.

Now, both placed side strips are pressed down and brought toward each other to evert the edges of the wound and mated by holding the side strips together, joining their respective zipper teeth, and inserting at one end the joined zipper teeth into the zipper slider, and then traversing the zipper slider to the other end and stopping the zipper slider there, which keeps the two side strips mated, resulting in wound closure.

To reinspect and/or reoperate on the wound, the zipper slider is traversed back to disengage the zipper teeth, which will provide an open access to the wound. And to reclose the wound, both placed side strips are re-mated as mentioned above.

An important advantage of the present invention is that it does not use sutures for wound closing, which require needles that carry a risk of injury to the patient and/or the practitioner, nor does it use staples for wound closing, which are often painful or traumatic to remove them after the wound has healed. Therefore, it will not cause all the other complications that are associated specifically with sutures or staples.

Another important advantage of the present invention is that it does not use tissue adhesives for wound closing, nor does it use adhesive tapes in a manner they are used for wound closing, which is to place multiple adhesive tapes across a wound and use cross adhesive tapes to support the adhesive tapes. Therefore, it will not cause all the complications that are associated specifically with tissue adhesives or adhesive tapes.

Yet, another important advantage of the present invention is that it will often not require anesthetics for closing certain wounds. Therefore, it will not cause all the complications that are associated specifically with anesthetics.

Still another important advantage of the present invention is that it will be less painful to the patient during its application for wound closing and less traumatic to the patient during its removal after wound healing.

Still another important advantage of the present invention is that it will allow the practitioner to easily and quickly inspect or reinspect the wound and/or to have an open access to reoperate on the wound and then easily and quickly reclose the wound by re-mating the already-placed side strips without any surgical tool. This will save time for the practitioner and money for the patient.

Still another important advantage of the present invention is that it will not require certain disposable or reusable accessories that are needed specifically for sutures, staples, tissue adhesives, or adhesive tapes, which will help relieve the workload burden on medical personnel and also will help reduce the costly biohazard disposal.

These and other advantages of the present invention will become apparent to those skilled in the art after a reading of the following detailed disclosure of embodiments of the present invention.

### BRIEF DESCRIPTION OF THE VIEWS IN THE DRAWING

### The First Embodiment

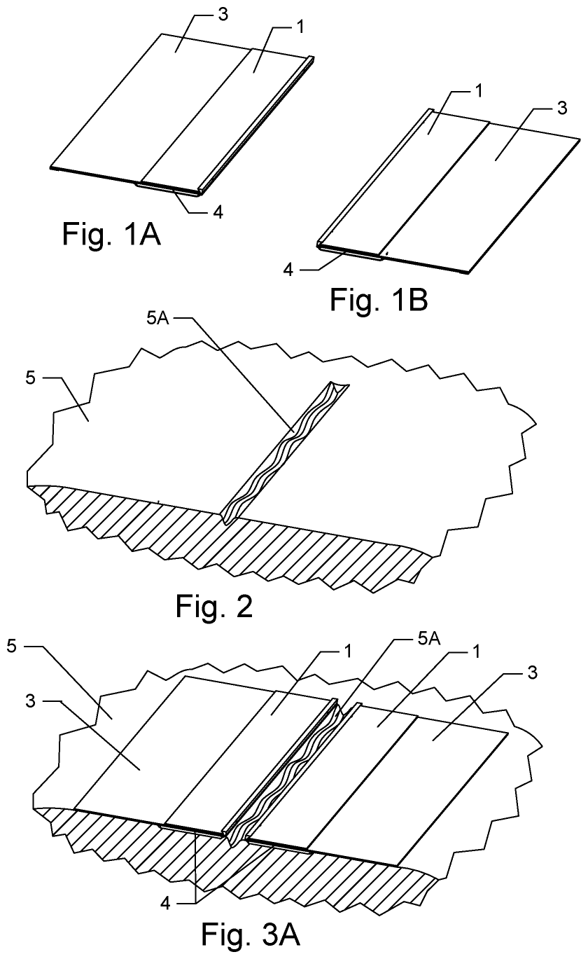

FIG. 1A and FIG. 1B show isometric views of the two side strips each attached to the custom bandage.

FIG. 2 shows an isometric cut-away view of a body part with a linear open wound.

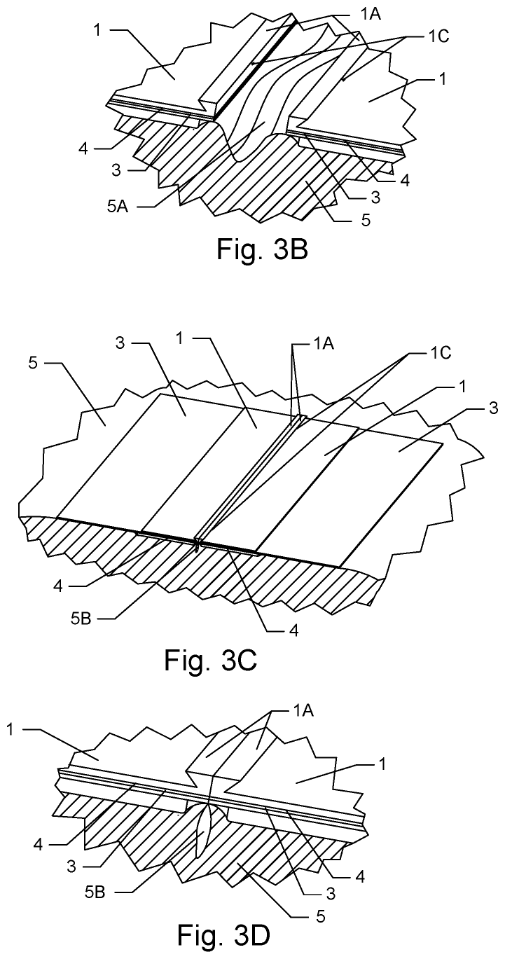

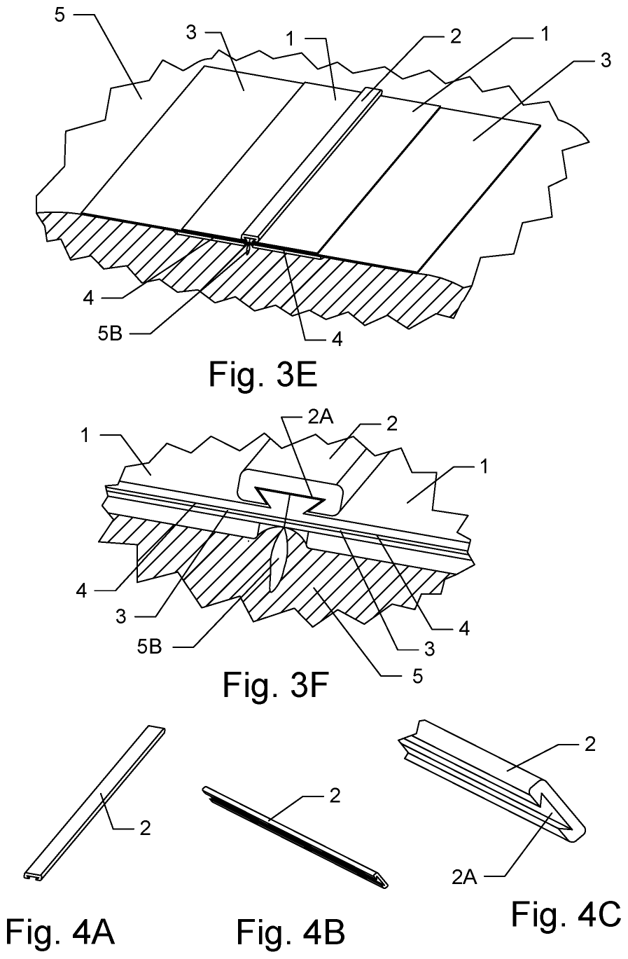

FIG. 3A shows an isometric view of the placement of the side strip on either side of the wound on the body part, while FIG. 3B shows an enlarged isometric cut-away view of the details around the area at the proximal end of the open wound.

FIG. 3C shows an isometric view of the placed side strips that are mated, while FIG. 3D shows an enlarged isometric cut-away view of the details around the area at the proximal end of the closed wound due to mating of the side strips.

FIG. 3E shows an isometric view of the two mated side strips that are captivated and securely locked in place by the installed slider strip, while FIG. 3F shows an enlarged isometric cut-away view of the details around the area at the proximal end of the closed wound and the mated side strips and the installed slider strip.

FIG. 4A and FIG. 4B show isometric views at two different rotational angles of the slider strip, while FIG. 4C shows an enlarged isometric cut-away view of the details around one end of the slider strip.

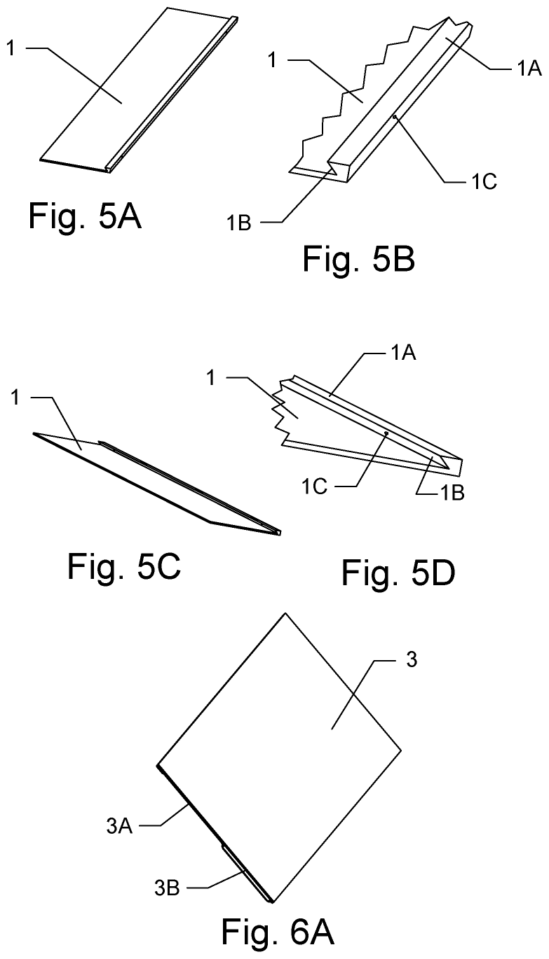

FIG. 5A and FIG. 5C show isometric views at two different rotational angles of the side strip, while FIG. 5B and FIG. 5D show, respectively, enlarged isometric cut-away views of the details around a corner of the side strip.

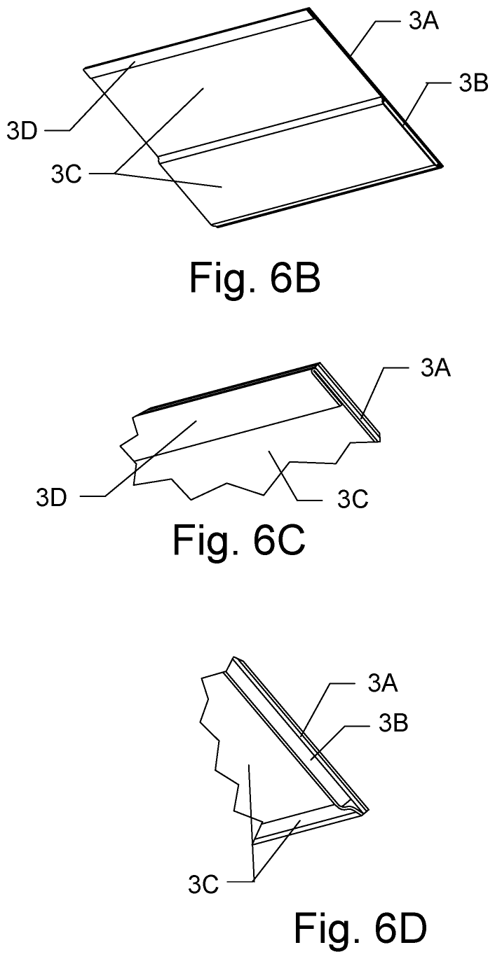

FIG. 6A and FIG. 6B show isometric views at two different rotational angles of the custom bandage, while FIG. 6C and FIG. 6D show, respectively, enlarged isometric cut-away views of the details around a corner of the custom bandage.

### The Second Embodiment

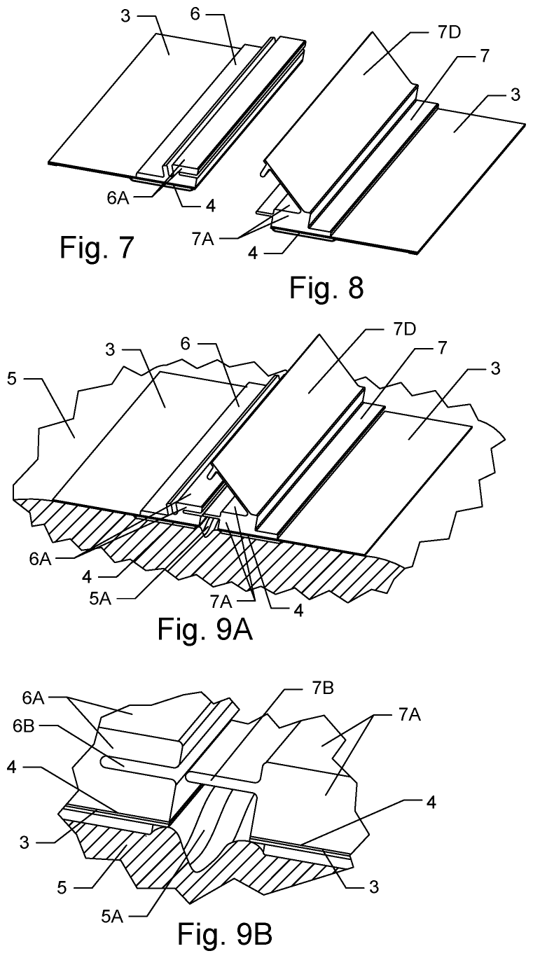

FIG. 7 shows an isometric view of the first side strip that has the receiving protrusion and that is attached to the custom bandage, while FIG. 8 shows an isometric view of the second side strip that has the latching protrusion and is also attached to the custom bandage.

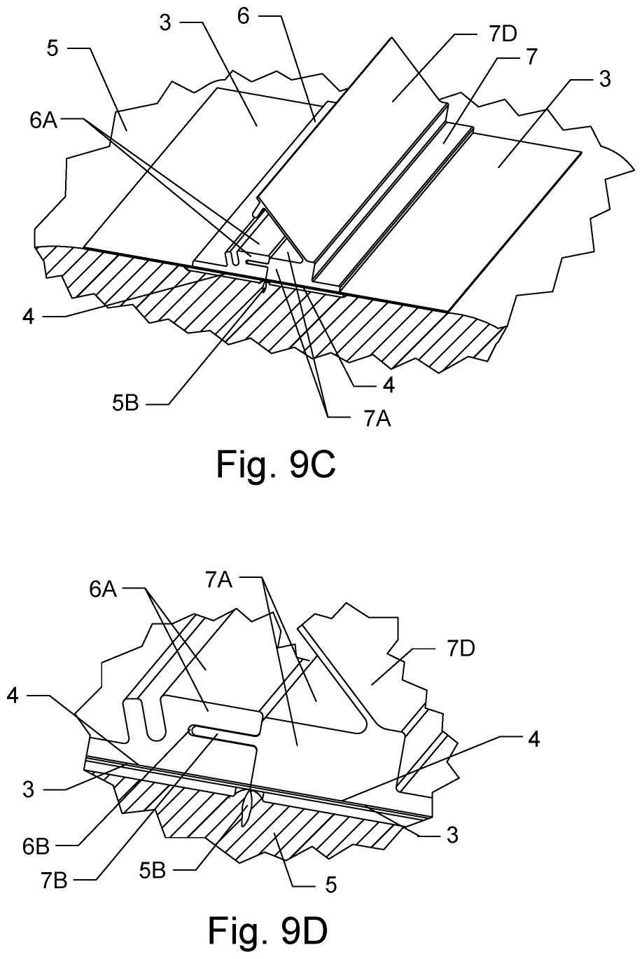

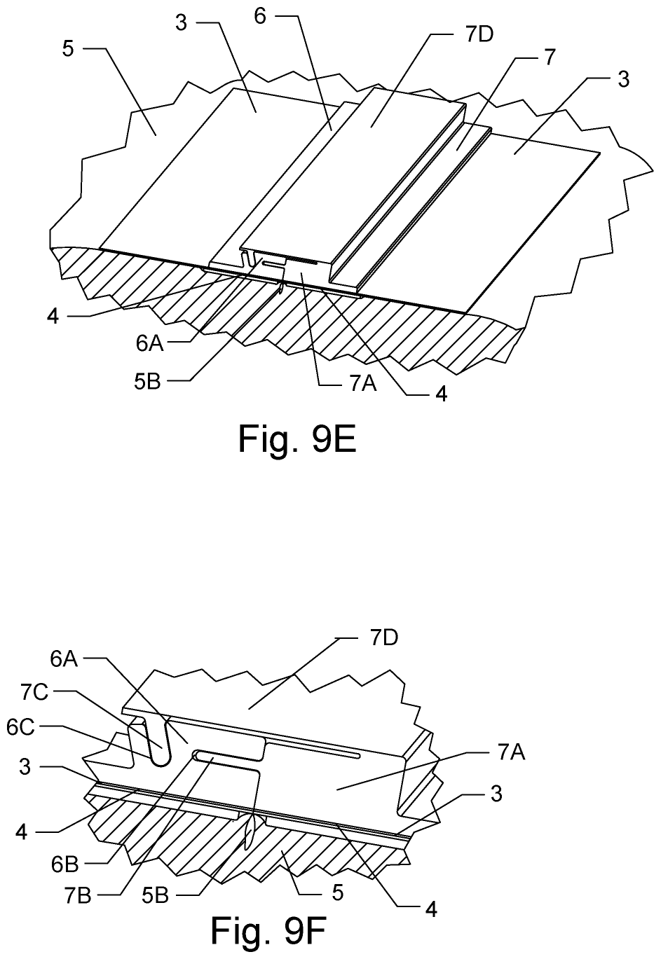

FIG. 9A shows an isometric view of the placement of the two side strips, each on either side of the wound on the body part, while FIG. 9B shows an enlarged isometric cut-away view of the details around the area at the proximal end of the open wound.

FIG. 9C shows an isometric view of the placed side strips that are mated, while FIG. 9D shows an enlarged isometric cut-away view of the details around the area at the proximal end of the closed wound due to mating of the side strips.

FIG. 9E shows an isometric view of the two mated side strips that are captivated and securely locked in place by the flap latch, while FIG. 9F shows an enlarged isometric cut-away view of the details around the area at the proximal end of the closed wound and the mated side strips that are captivated and locked in place by the flap latch.

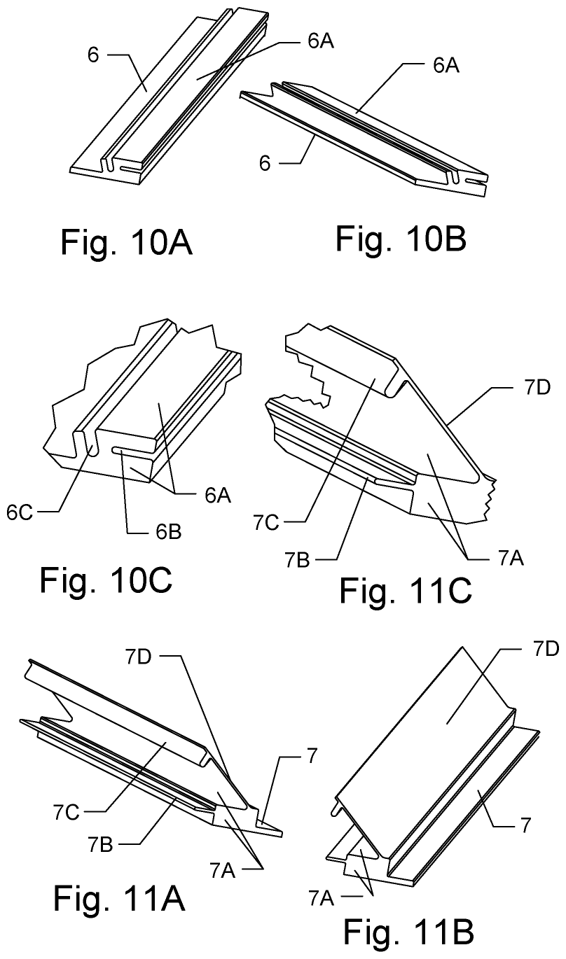

FIG. 10A and FIG. 10B show isometric views at two different rotational angles of the side strip that has the angled slot on the top and the straight slot on the side, while FIG. 10C shows an enlarged isometric cut-away view at different rotational angles of the details around a corner of the side strip.

FIG. 11A and FIG. 11B show isometric views at two different rotational angles of the side strip that has the flap latch and the straight rib, while FIG. 11C shows an enlarged isometric cut-away view of the details around a corner of the side strip.

### The Third Embodiment

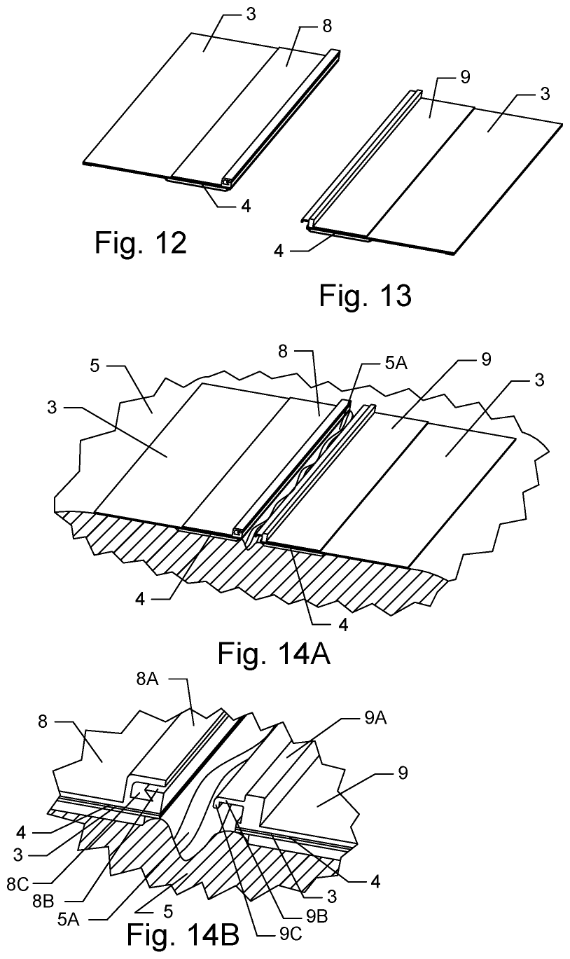

FIG. 12 shows an isometric view of the side strip that has the G-receptacle protrusion and that is attached to the custom bandage, while FIG. 13 shows an isometric view of the other side strip that has the engaging protrusion and that is also attached to the custom bandage.

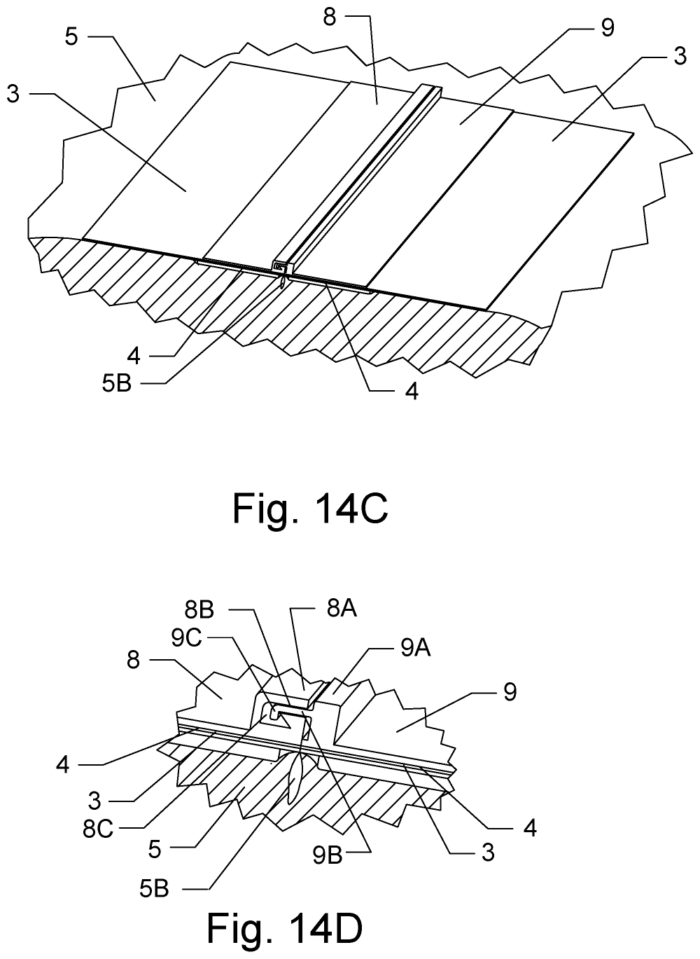

FIG. 14A shows an isometric view of the placement of the two side strips, each on either side of the wound on the body part, while FIG. 14B shows an enlarged isometric cut-away view of the details around the area at the proximal end of the open wound.

FIG. 14C shows an isometric view of the placed side strips that are mated, captivated, and locked in place, while FIG. 14D shows an enlarged isometric cut-away view of the details around the area at the proximal end of the closed wound due to mated, captivated, locked side strips.

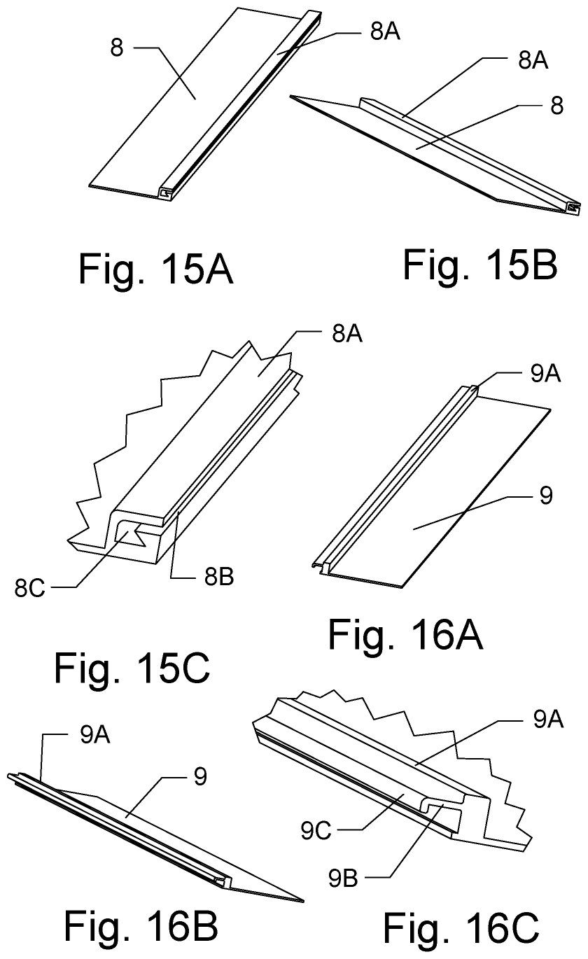

FIG. 15A and FIG. 15B show isometric views at two different rotational angles of the side strip that has the G-receptacle protrusion, while FIG. 15C shows an enlarged isometric cut-away view of the details around a corner of the side strip.

FIG. 16A and FIG. 16B show isometric views at different rotational angles of the side strip that has the engaging protrusion while FIG. 16C shows an enlarged isometric cut-away view of the details around a corner of the side strip.

### The Fourth Embodiment

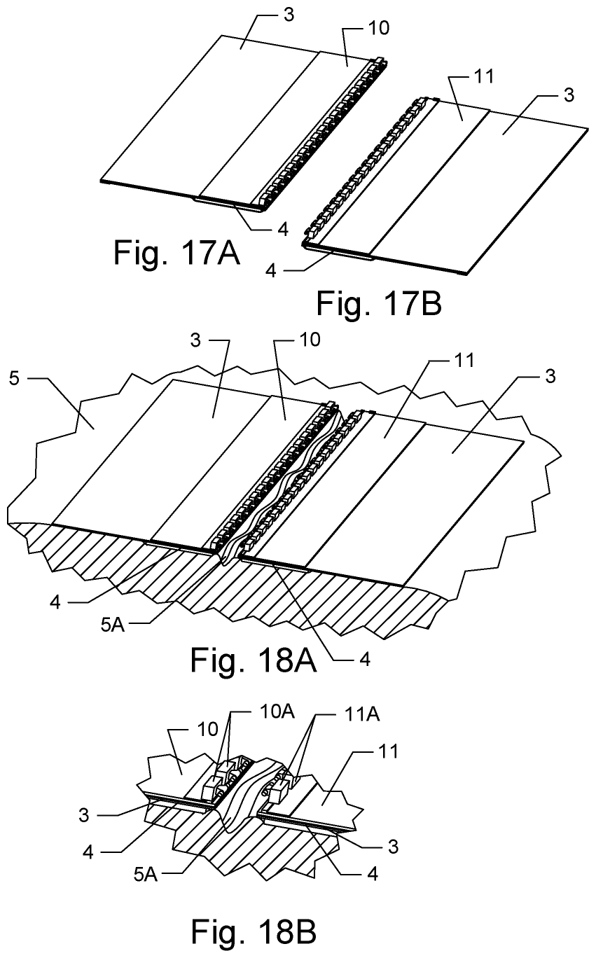

FIG. 17A shows an isometric view of the side strip that has the zipper-teeth chain and that is attached to the custom bandage, while FIG. 17B shows an isometric view of the other side strip that has the matching zipper-teeth chain and that is also attached to the custom bandage.

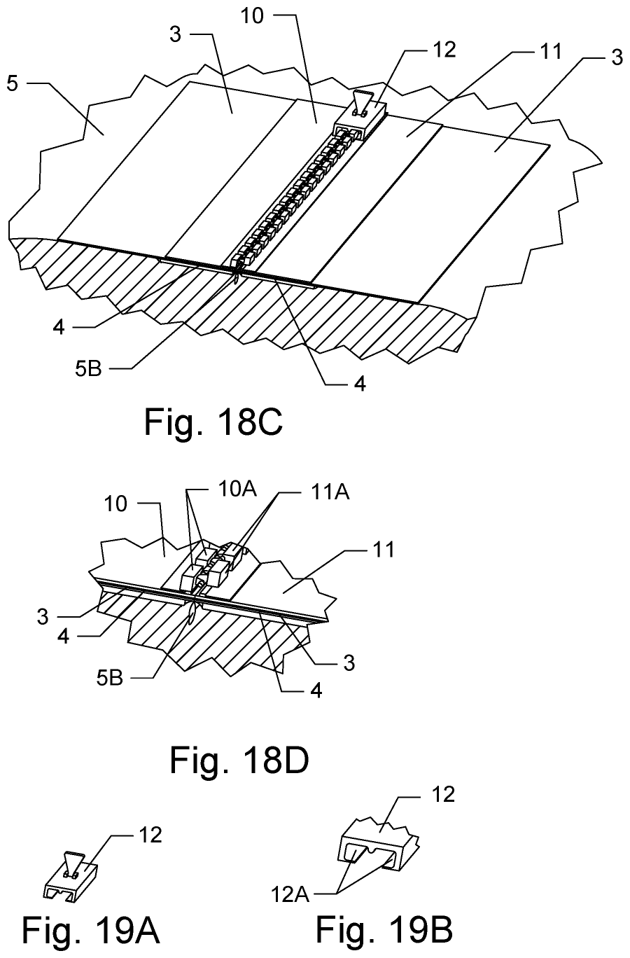

FIG. 18A shows an isometric view of the placement of the two side strips, each on either side of the wound on the body part, while FIG. 18B shows an enlarged isometric cut-away view of the details around the area at the proximal end of the open wound.

FIG. 18C shows an isometric view of the placed side strips that are mated, captivated, and locked in place by the zipper slider, while FIG. 18D shows an enlarged isometric cut-away view of the details around the area at the proximal end of the closed wound due to mated, captivated, locked side strips.

FIG. 19A shows an isometric view of the zipper slider, while FIG. 19B shows an enlarged isometric cut-away view of the details around one end of the zipper slider.

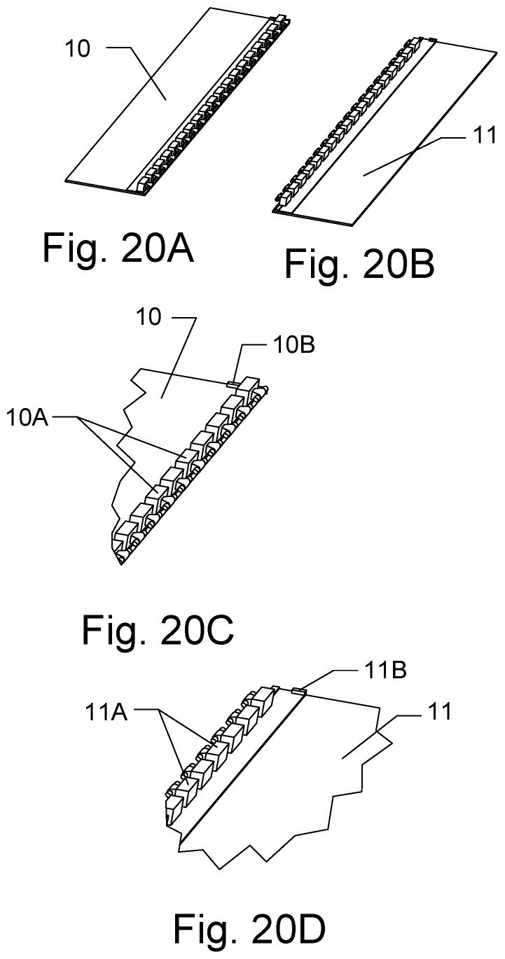

FIG. 20A and FIG. 20B show isometric views of two side strips each with the zipper teeth chain, while FIG. 20C and FIG. 20D show, respectively, enlarged isometric cut-away views of the details around a corner of the two side strips.

### DETAILED DESCRIPTION OF THE EMBODIMENTS

### The First Embodiment

Now, referring to FIG. 1A, FIG. 1B, FIGS. 4A, FIG. 4B, FIG. 4C, FIG. 5A, FIG. 5B, FIG. 5C, and FIG. 5D, which show isometric views of a first embodiment of the non-invasive wound closure device in accordance with the present invention, The wound closure device consists of a side strip 1 in a quantity of at least two, and a custom bandage 3.

The side strip 1 on its bottom planar surface is attached or integral via a mechanical, thermal, electrical, chemical, ultrasonic, photonic, adhesive, or any other bonding or forming means 4 to the top non-adhering planar surface of the custom bandage 3 and contains on the other planar surface of the side strip 1 an offset protrusion 1A longitudinally along the entire length of the side strip 1. The protrusion 1A has at least two side holes 1C and an inwardly angled side wall 1B in order for the protrusion 1A to form a wedge tongue when the protrusion 1A of one side strip 1 is aligned with the protrusion 1A of the other side strip 1 by matching manually or via a tool the holes 1C and when the protrusion 1A of one side strip 1 is brought together with the protrusion 1A of the other side strip 1.

A slider strip 2, longitudinally along its entire length, has an end-to-end open, transversely blind slot 2A whose side walls are also inwardly angled so as to match the inwardly angled side wall 1B of the protrusion 1A of the side strip 1 and to form a wedge groove.

Referring to FIG. 6A, FIG. 6B, FIG. 6C, and FIG. 6D, which show isometric views, the custom bandage 3 is a modified version of a typical bandage that is normally used for covering wounds or skin cuts and that has an adhesive tape 3A, a piece of a fabric absorbent 3B, and a peel-away backing 3C. The modification is made to lengthen the fabric absorbent 3B to the length of the side strip 1 and to offset the fabric absorbent 3B to one side so that the offset fabric absorbent 3B is longitudinally continuous from one end to the other end of the custom bandage 3. For its easier removal, the peel-away backing 3C has a folded end 3D.

Referring to FIG. 2, FIG. 3A, and FIG. 3B, which show isometric views, a body part 5 has an open wound 5A that is linear. To reapproximate the open wound 5A that has been cleaned and medicinally prepped, the backing 3C of the custom bandage that is integral to the side strip 1 is peeled off from the folded end 3D and then the side strip 1 with the custom bandage 3 that is now without the backing 3C is placed on one side of the open wound 5A while making sure that the protrusion 1A of the side strip 1 is parallel and adjacent to the edge of the open wound 5A and that the custom bandage 3 of the side strip 1 is properly adhered to the skin of the body part 5 by pressing down on the side strip 1 with a swiping finger or thump. For the other side of the open wound 5A, the same is done using the other side strip 1 with the custom bandage 3 while making sure that the protrusion 1A of the side strip 1 with the custom bandage 3 faces the protrusion 1A of the other side strip 1 with the custom bandage 3 that was placed before.

Also, referring to FIG. 3C, FIG. 3D, FIG. 3E, and FIG. 3F, which show isometric views, now the placed side strip 1 with the custom bandage 3 is pressed down and brought toward the other placed side strip 1 with the custom bandage 3 in order to evert the edges of the open wound 5A and the two side strips 1, each with the custom bandage 3 are mated by aligning and joining the protrusions 3A together manually or by temporarily inserting an appropriate pin into the alignment holes 1C of the protrusions 1A; and then while holding the aligned protrusions 1A together, the slider strip 2 is installed by inserting the aligned and joined protrusions 1A, which together form the wedge tongue, into the slot 2A of the slider strip 2, which slot acts as the wedge groove, and by sliding the slider strip 2 on to the aligned and joined protrusions 1A all the way so as to fully captivate the joined protrusions 1A and to securely lock in place the mated side strips 1, each with the custom bandage 3, thereby resulting in a closed wound 5B of the body part 5.

Later, to reinspect the closed wound 5B and/or to reoperate on the reopened wound 5A, the slider strip 2 is slid out, which will separate the mated side strips 1, each with the custom bandage 3, and which thereby will provide an open access to the closed wound 5B. or the reopened wound 5A. And to reclose the closed wound 5B or the reopened wound 5A, both placed side strips 1, each with the custom bandage 3, are re-mated as mentioned above.

### The Second Embodiment

Now, referring to FIG. 7, FIG. 8, FIGS. 10A, FIG. 10B, FIG. 10C, FIG. 11A, FIG. 11B, and FIG. 11C, which show isometric views of a second embodiment of the non-invasive wound closure device in accordance with the present invention, The wound closure device consists of a first side strip 6 and a second side strip 7 and a custom bandage 3.

The first side strip 6 on its bottom planar surface is attached or integral via a mechanical, thermal, electrical, chemical, ultrasonic, photonic, adhesive, or any other bonding or forming means 4 to the top non-adhering planar surface of the custom bandage 3 and contains on the other planar surface of the first side strip 6 an offset faceted receiving protrusion 6A longitudinally along the entire length of the first side strip 6. Also, longitudinally along its entire length, the receiving protrusion 6A of the first side strip 6 has an angled slot 6C on the top and a straight slot 6B on the side.

The second side strip 7 on its bottom planar surface is attached or integral via a mechanical, thermal, electrical, chemical, ultrasonic, photonic, adhesive, or any other bonding or forming means 4 to the top non-adhering planar surface of the custom bandage 3 and contains on the other planar surface of the second side strip 7 an offset faceted latching protrusion 7A longitudinally along the entire length of the second side strip 7. Also, longitudinally along its entire length, the latching protrusion 7A of the second side strip 7 contains a flap latch 7D that is flexible at its longitudinal joining end with the latching protrusion 7A in order to act as a natural or living hinge and that has an angled rib 7C, on and near the free end, longitudinally along the entire length, of the flap latch. The latching protrusion 7A also contains a straight rib 7B on one side.

The angled slot 6C of the receiving protrusion 6A is for the angled rib 7C of the flap latch 7D while the straight slot 6B of the receiving protrusion 6A is for the straight rib 7B of the latching protrusion 7A.

Referring to FIG. 2, FIG. 9A, and FIG. 9B, which show isometric views, to reapproximate the open wound 5A of the body part 5, which open wound 5A has been cleaned and medicinally prepped, the backing 3C of the custom bandage that is integral to the first side strip 6 is peeled off from the folded end 3D and then the first side strip 6 with the custom bandage 3 that is now without the backing 3C is placed on one side of the open wound 5A while making sure that the receiving protrusion 6A of the first side strip 6 is parallel and adjacent to the edge of the open wound 5A and that the custom bandage 3 of the first side strip 6 is properly adhered to the skin of the body part 5 by pressing down on the first side strip 6 with a swiping finger or thump. For the other side of the open wound 5A, the same is done using the second side strip 7 with the custom bandage 3 while making sure that the latching protrusion 7A of the second side strip 7 with the custom bandage 3 faces the receiving protrusion 6A of the first side strip 6 with the custom bandage 3 that was placed before.

Also, referring to FIG. 9C, FIG. 9D, FIG. 9E, and FIG. 9F, which show isometric views, now, the placed first side strip 6 with the custom bandage 3 is pressed down and brought toward the placed second side strip 7 with the custom bandage 3 in order to evert the edges of the open wound 5A and the first side strip 6 and the second side strip 7, each with the custom bandage 3, are mated by aligning and joining the receiving protrusion 6A and the latching protrusion 7A together manually while fully inserting the straight rib 7B of the latching protrusion 7A into the corresponding straight slot 6B of the receiving protrusion 6A; and then while holding the mated first side strip 6 and second side strip 7 together, the flap latch 7D of the latching protrusion 7A is placed by inserting the angled rib 7C of the flap latch 7D of the latching protrusion 7A into the angled slot 6C of the receiving protrusion 6A. By doing so captivates and securely locks in place the mated first side strip 6 and second side strip 7, and thereby results in a closed wound 5B of the body part 5.

Later, to reinspect the closed wound 5B and/or to reoperate on the reopened wound 5A, the flap latch 7D is lifted to disengage it, which will separate the mated first side strip 6 and second side strip 7, each with the custom bandage 3, and which thereby will provide an open access to the closed wound 5B. or the reopened wound 5A. And to reclose the closed wound 5B or the reopened wound 5A, both the placed first side strip 6 and second side strip 7, each with the custom bandage 3, are re-mated as mentioned above.

### The Third Embodiment

Now, referring to FIG. 12, FIG. 13, FIGS. 15A, FIG. 15B, FIG. 15C, FIG. 16A, FIG. 16B, and FIG. 16C, which show isometric views of a third embodiment of the non-invasive wound closure device in accordance with the present invention, The wound closure device consists of a first side strip 8 and a second side strip 9 and a custom bandage 3. The first side strip 8 on its bottom planar surface is attached or integral via a mechanical, thermal, electrical, chemical, ultrasonic, photonic, adhesive, or any other bonding or forming means 4 to the top non-adhering planar surface of the custom bandage 3 and longitudinally contains on the other planar surface of the first side strip 8 along its entire length an offset G-receptacle protrusion 8A that has a cross-sectional shape, which looks like the capital letter G and that due to the G shape of the cross section, has a linear opening 8B as a receiving feature and a linear wedge 8C inside as a locking feature, longitudinally along the entire length of the G-receptacle protrusion 8A.

The second side strip 9 on its bottom planar surface is attached or integral via a mechanical, thermal, electrical, chemical, ultrasonic, photonic, adhesive, or any other bonding or forming means 4 to the top non-adhering planar surface of the custom bandage 3 and contains on the other planar surface of the second side strip 9 an offset engaging protrusion 9A longitudinally along the entire length of the second side strip 9. Longitudinally along its entire length, the engaging protrusion 9A has on its one side an extended rib 9B with a flanged end 9C.

The extended rib 9B with the flanged end 9C of the engaging protrusion 9A goes into the linear opening 8B of the G-receptacle protrusion 8A and locks into the linear wedge 8C of the G-receptacle protrusion 8A.

Referring to FIG. 2, FIG. 14A, and FIG. 14B, which show isometric views, in order to reapproximate the open wound 5A of the body part 5, which open wound 5A has been cleaned and medicinally prepped, the backing 3C of the custom bandage that is integral to the first side strip 8 is peeled off from the folded end 3D and then the first side strip 8 with the custom bandage 3 that is now without the backing 3C is placed on one side of the open wound 5A while making sure that the G-receptacle protrusion 8A of the first side strip 8 is parallel and adjacent to the edge of the open wound 5A and that the custom bandage 3 of the first side strip 8 is properly adhered to the skin of the body part 5 by pressing down on the first side strip 8 with a swiping finger or thump. For the other side of the open wound 5A, the same is done using the second side strip 9 with the custom bandage 3 while making sure that the engaging protrusion 9A of the second side strip 9 with the custom bandage 3 faces the G-receptacle protrusion 8A of the first side strip 8 with the custom bandage 3 that was placed before.

Also, referring to FIG. 14C and FIG. 14D, which show isometric views, now, the placed first side strip 8 with the custom bandage 3 is pressed down and brought toward the placed second side strip 9 with the custom bandage 3 in order to evert the edges of the open wound 5A and the first side strip 8 and the second side strip 9, each with the custom bandage 3, are mated by aligning and joining the G-receptacle protrusion 8A and the engaging protrusion 9A together manually while fully inserting all the way the extended rib 9B of the engaging protrusion 9A into the corresponding linear opening 8B of the G-receptacle protrusion 8A while ensuring that the flanged end 9C. of the extended rib 9B of the engaging protrusion 9A locks into the linear wedge 8C of the of the G-receptacle protrusion 8A. By doing so captivates and securely locks in place the mated first side strip 8 and the second side strip 9, and thereby results in a closed wound 5B of the body part 5.

Later, to reinspect the closed wound 5B and/or to reoperate on the reopened wound 5A, the linear opening 8B of the receptacle protrusion 8A is widened with fingers or by using an appropriate prying pin to disengage the extended rib 9B, which will separate the mated first side strip 8 and second side strip 9, each with the custom bandage 3, and which thereby will provide an open access to the closed wound 5B. or the reopened wound 5A. And to reclose the closed wound 5B or the reopened wound 5A, both the placed first side strip 8 and second side strip 9, each with the custom bandage 3, are re-mated as mentioned above.

### The Fourth Embodiment

Now, referring to FIG. 17A, FIG. 17B, FIGS. 19A, FIG. 19B, FIG. 20A, FIG. 20B, FIG. 20C, and FIG. 20D, which show isometric views of a fourth embodiment of the non-invasive wound closure device in accordance with the present invention, The wound closure device consists of a side strip 10 and an opposing side strip 11, which is slightly different than the side strip 10, a zipper slider 12, and a custom bandage 3. The side strip 10 and the opposing side strip 11 on their respective bottom planar surface are individually attached or integral via a mechanical, thermal, electrical, chemical, ultrasonic, photonic, adhesive, or any other bonding or forming means 4 to the top non-adhering planar surface of the custom bandage 3. The side strip 10 contains on its other planar surface an offset zipper slider stop 10B at one end and an offset and welded or bonded zipper-teeth chain 10A longitudinally along the entire length of the side strip 10, while the opposing side strip 11 contains also on its other planar surface an offset matching zipper slider stop 11B and an offset and welded or bonded matching zipper-teeth chain 11A, longitudinally along the entire length of the opposing side strip 11.

Both the zipper-teeth chain 10A of the side strip 10 and the matching zipper-teeth chain 11A of the opposing side strip 11 are standard zippers and the zipper slider 12 that engages and interlocks the zipper teeth is also standard.

Referring to FIG. 2, FIG. 18A, and FIG. 18B, which show isometric views, in order to reapproximate the open wound 5A of the body part 5, which open wound 5A has been cleaned and medicinally prepped, the backing 3C of the custom bandage that is integral to the side strip 10 is peeled off from the folded end 3D and then the side strip 10 with the custom bandage 3 that is now without the backing 3C is placed on one side of the open wound 5A while making sure that the zipper-teeth chain 10A of the side strip 10 is parallel and adjacent to the edge of the open wound 5A and that the custom bandage 3 of the side strip 10 is properly adhered to the skin of the body part 5 by pressing down on the side strip 10 with a swiping finger or thump. For the other side of the open wound 5A, the same is done using the opposing side strip 11 with the custom bandage 3 while making sure that the matching zipper-teeth chain 11A of the opposing side strip 11 with the custom bandage 3 faces the zipper-teeth chain 10A of the side strip 10 with the custom bandage 3 that was placed before.

Also, referring to FIG. 18C and FIG. 18D, which show isometric views, now, the placed side strip 10 with the custom bandage 3 is pressed down and brought toward the placed opposing side strip 11 with the custom bandage 3 in order to evert the edges of the open wound 5A and the side strip 10 and the opposing side strip 11, each with the custom bandage 3, are mated by aligning and joining the zipper-teeth chain 10A and the matching zipper-teeth chain 11A together while manually inserting at one end the joined zipper teeth into the opening 12A of the zipper slider 12, and then traversing the zipper slider 12 all the way toward the zipper slider stop 10B of the side strip 10 and the matching zipper slider stop 11B of the opposing side strip 11 in order to interlock the zipper-teeth chain 10A and the matching zipper-teeth chain 11A. The zipper slider 12 is left in place at the other end of the interlocked zipper-teeth chain 10A. and the matching zipper-teeth chain 11A. By doing so captivates and securely locks in place the mated side strip 10 and opposing side strip 11 and thereby results in a closed wound 5B of the body part 5.

Later, to reinspect the closed wound 5B and/or to reoperate on the reopened wound 5A, the zipper slider 12 is traversed back to disengage the interlocked zipper teeth chain 10A and the matching zipper-teeth chain 11A, which will separate the mated side strip 10 and opposing side strip 11, each with the custom bandage 3, and which thereby will provide an open access to the closed wound 5B. or the reopened wound 5A. And to reclose the closed wound 5B or the reopened wound 5A, both the placed side strip 10 and opposing side strip 11, each with the custom bandage 3, are re-mated as mentioned above.

The invention claimed is:

1. A non-invasive wound closure device, to reapproximate the everted edges of an injury wound or surgical cut of a patient, having two elongated side strips each adhered to either side of the wound or cut that is closed by manually mating and mechanistically interlocking said side strips, that can also be reopened when needed by mechanistically unlocking and manually separating the mated said side strips, and that can be then reclosed by repeating the closure process, said device comprising:
an elongated protrusion on a top surface of said side strip longitudinally along entire length thereof, said protrusion having a flat face on a side surface thereof and an angled or straight under-cut wedge face on other side surface thereof to form an elongated wedge tongue longitudinally along entire length thereof, and having at least two cross holes separated and located longitudinally on said flat face;
an elongated slider strip having on a surface thereof an elongated transversely blind slot with open ends longitudinally along entire length thereof to form an elongated angled or straight under-cut groove to match said tongue of said side strip; and
an elongated customized conventional bandage having an polymer or fabric layer wherein a top surface thereof is attached to a bottom surface of said side strip, and said bandage having on a bottom surface thereof a fully-covering adhesive layer capable of adhering to patient's skin, a partially-covering fluid-absorbent fabric or polymer layer, and a fully-covering peel-away backing layer made of a polymer or any other material, all longitudinally along entire length of said bandage.

2. A non-invasive wound closure device, to reapproximate the everted edges of an injury wound or surgical cut of a patient, having two different elongated side strips each integrally containing said bandage adhered to either side of the wound or cut that is closed by manually mating and mechanistically interlocking said side strips, that can also be reopened when needed by mechanistically unlocking and manually separating the mated said side strips, and that can be then reclosed by repeating the closure process, said device comprising:
an elongated protrusion on a top surface of said side strip longitudinally along entire length thereof, said protrusion having on a top surface thereof an elongated and angled blind slot with open ends longitudinally along entire length thereof and having on a side surface thereof an elongated and laterally straight blind slot with open ends longitudinally along entire length thereof; and
an elongated protrusion on a top surface of another said side strip longitudinally along entire length thereof, said protrusion having on a side surface thereof an elongated laterally straight rib longitudinally along entire length thereof and having on a top surface thereof an elongated integral flex-hinged flap latch longitudinally along entire length thereof, and said latch having at a free end thereof an elongated slightly offset angled rib longitudinally along entire length thereof.

3. A non-invasive wound closure device, to reapproximate the everted edges of an injury wound or surgical cut of a patient, having two different elongated side strips each integrally containing said bandage adhered to either side of the wound or cut that is closed by manually mating and mechanistically interlocking said side strips, that can also be reopened when needed by mechanistically unlocking and manually separating the mated said side strips, and that can be then reclosed by repeating the closure process, said device comprising:
an elongated protrusion on a top surface of said side strip longitudinally along entire length thereof, said protrusion having a hollow cross-sectional shape mimicking a capital letter G wherein said protrusion has an elongated internal empty space with an elongated internal angled or straight under-cut wedge surface and has on an external side surface thereof an elongated opening with clear ends as a pathway to said space longitudinally along entire length thereof; and.
an elongated protrusion on a top surface of another said side strip longitudinally along entire length thereof, said protrusion having on a side surface thereof an elongated and laterally a straight rib with an elongated flanged end longitudinally along entire length thereof.

4. A non-invasive wound closure device, to reapproximate the everted edges of an injury wound or surgical cut of a patient, having two slightly different elongated side strips each integrally containing said bandage adhered to either side of the wound or cut that is closed by manually mating and mechanistically interlocking said side strips, that can also be reopened when needed by mechanistically unlocking and manually separating the mated said side strips, and that can be then reclosed by repeating the closure process, said device comprising:
an elongated standard zipper-teeth chain integrally on a top surface of said side strip longitudinally along entire length thereof, said side strip having on a top surface thereof a boss of a round, elliptical, triangular, trapezoidal, square, rectangular, or any other geometric or organic shape at or near an end of said chain;
an elongated matching staggered standard zipper-teeth chain integrally on top surface of other said side strip longitudinally along entire length thereof, said side strip having on a top surface thereof a boss of a round, elliptical, triangular, trapezoidal, square, rectangular, or any other geometric or organic shape at or near an end of said chain; and
a standard zipper slider for interlocking said chain and said matching chain, said slider having on a bottom surface thereof a standard opening wherein said chain and said matching chain are inserted, joined together, and interlocked by traversing and stopping said slider at said boss.
